(19) United States
(12) Patent Application Publication
(10) Pub. No.: US 2024/0033507 A1
(43) Pub. Date: Feb. 1, 2024

(54) IMPLANTABLE SUPPORT FOR MEDICAL IMPLANT

(71) Applicant: Cochlear Limited, Macquarie University, New South Wales (AU)

(72) Inventors: Wim Bervoets, Mechelen (BE); Olivier Le Caharec, Mechelen (BE)

(21) Appl. No.: 18/256,327

(22) PCT Filed: Dec. 27, 2021
(86) PCT No.: PCT/IB2021/062349

§ 371 Date: Jun. 7, 2023

Related U.S. Application Data
(60) Provisional application No. 63/133,598, filed on Jan. 4, 2021.

(51) Int. Cl. A61N 1/05; A61N 1/36; A61N 1/375

(52) U.S. Cl. CPC A61N 1/0541; A61N 1/36039; A61N 1/375

(57) ABSTRACT

An apparatus includes a first portion configured to be implanted at a first location on or within a recipient's body and a second portion configured to be implanted on or within the recipient's body and configured to be mechanically coupled to a transducer and/or reservoir. The second portion includes an orifice and an elongate element extending through the orifice. The elongate element is configured to be in mechanical communication with a second location on or within the recipient's body, the second location spaced from the first portion. The first portion and the second portion are configured to be implanted with the elongate element in mechanical communication with the second location prior to the second portion being operatively coupled to the transducer and/or the reservoir.

### BACKGROUND

### Field

The present application relates generally to medical implants (e.g., implantable medical prostheses) having active components (e.g., transducers; actuators; microphones; reservoirs of liquid medicament).

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a first portion configured to be implanted at a first location on or within a recipient's body and a second portion configured to be implanted on or within the recipient's body and configured to be mechanically coupled to a transducer. The second portion comprises an orifice and an elongate element extending through the orifice. The elongate element is configured to be in mechanical communication with a second location on or within the recipient's body, the second location spaced from the first portion. The first portion and the second portion are configured to be implanted with the elongate element in mechanical communication with the second location prior to the second portion being mechanically coupled to the transducer.

In another aspect disclosed herein, an apparatus comprises a fixation portion configured to be implanted on or within a recipient's body and an elongate portion configured to be in mechanical communication with a transducer and a target portion of the recipient's body. The elongate portion extends at least partially through the fixation portion and is configured to transmit vibrational energy from the transducer to the target portion and/or from the target portion to the transducer. The apparatus further comprises at least one mechanically compliant portion between the fixation portion and the elongate portion. The at least one mechanically compliant portion is configured to substantially inhibit escape of the vibrational energy from the elongate portion to portions of the recipient's body spaced from the target portion and/or to substantially inhibit vibrational energy not from the target portion from reaching the transducer.

In another aspect disclosed herein, a method comprises at least partially implanting an assembly on or within a recipient's body. The assembly comprises a fixation portion and an elongate portion extending at least partially through the fixation portion. Said at least partially implanting comprises affixing the fixation portion to a first location of the recipient's body. The method further comprises, while the fixation portion is affixed to the first location, adjusting a position and/or orientation of the elongate portion to be in operative communication with a second location of the recipient's body. The method further comprises, while the elongate portion is in operative communication with the second location of the recipient's body, operatively coupling the elongate portion with a transducer and/or a reservoir configured to contain at least one medicament.

In another aspect disclosed herein, an apparatus comprises a fixation bracket configured to be affixed at a first location on or within a recipient's body and an elongate fluid conduit configured to be at least partially within the fixation bracket. The elongate fluid conduit comprises a first end portion configured to be in fluidic communication with a reservoir configured to contain fluid and a second end portion configured to be in fluidic communication with a second location on or within the recipient's body and spaced from the first location. The fixation bracket and the elongate fluid conduit are configured to be implanted on or within the recipient's body prior to the reservoir being in fluidic communication with the first end portion.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 is a perspective view of an example fully implantable middle ear implant auditory prosthesis implanted in a recipient in accordance with certain implementations described herein;

FIG. 3A schematically illustrates an example prior middle ear transducer assemblies that are based on a two-point fixation concept;

FIGS. 3B and 3C schematically illustrate a second end of a connection apparatus having a structure configured to be affixed to the ossicles in accordance with certain implementations described herein;

FIG. 4 schematically illustrates various types of vibration stimulation that can be provided to various structures in the cochleovestibular region in accordance with certain implementations described herein;

FIGS. 5A-5C schematically illustrate various example apparatus in accordance with certain implementations described herein;

FIGS. 6A-6F schematically illustrate various other example apparatus in accordance with certain implementations described herein;

FIG. 7 is a flow diagram of an example method in accordance with certain implementations described herein; and

FIGS. 8A-8E schematically illustrate various stages of an assembly in accordance with certain implementations described herein during the example method of FIG. 7.

### DETAILED DESCRIPTION

Sensorineural hearing loss (SNHL) is a permanent hearing loss due to damage that prevents or weakens nerve signals transmitted to the brain. Severe to profound SNHL can be addressed by a cochlear implant auditory prosthesis, while less severe SNHL can be addressed by an auditory prosthesis comprising a middle ear implant in contact with one of the ossicles of the ear, since patients with less severe SNHL can have a middle ear ossicular chain that is intact (e.g., capable of moving freely; not being blocked or having excessive conductive loss). Such middle ear implants follow the general practice of not removing functioning parts of the body unless necessary, and can be more effective for addressing less severe SNHL than are hearing aids or auditory prostheses using bone-anchored implants.

Conductive hearing loss (CHL) is due to obstruction or damage to the outer ear or middle ear that prevents sound from being conducted to the inner ear. CHL can be addressed by an auditory prosthesis comprising a bone-anchored hearing aid that transmits sound vibrations to travel through the skull bone to the inner ear, thereby bypassing the middle ear ossicles and tympanic membrane. Such bone-anchored hearing aids can be more effective for many CHL patients than are hearing aids that are positioned within the ear canal (e.g., which can utilize large amplification levels which can result in feedback issues).

Mixed hearing loss (MHL) is any combination of SNHL and CHL and can be addressed by an auditory prosthesis comprising a bone-anchored hearing aid or comprising a middle ear implant (e.g., to address the SNHL component). However, in contrast to addressing solely SNHL, addressing MHL can comprise removal and replacement or bypass (e.g., by extension or prosthesis) of part of the ossicular chain to address the CHL component.

Certain implementations described herein provide an adjustable (e.g., sliding; threaded; screw-like) extension configured to be implanted and mechanically coupled to a target location before an active component (e.g., transducer and/or reservoir) of a medical implant assembly is implanted. The extension can be mechanically coupled to the target location before being mechanically coupled to the active component. Certain such implementations reduce the number of components to be manipulated (e.g., positioned) by the practitioner performing the implantation and/or provides the practitioner with improved (e.g., maximum) visibility to the target location, thereby facilitating increased speed, increased ease, reduced risk of failure and/or damage to the implant and/or target location, and more consistent and reliable outcomes (e.g., successful coupling between the implant and the target location).

Certain implementations described herein include vibration isolation configured to prevent actuator vibrations from reaching the target location via other pathways separate from the extension. Certain such implementations provide more effective vibration and prevention of parallel vibration paths to the target location (e.g., canalizing the vibrations to the target location), thus preventing harmonic distortions (e.g., for high-power transducers for stimulating a high impedance stimulation target, such as the otic capsule, in a two-point fixation configuration), for more efficient stimulation and improved sound quality.

The teachings detailed herein are applicable, in at least some implementations, to any type of auditory prosthesis utilizing an implantable transducer assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. In some implementations, the teachings detailed herein and/or variations thereof can be utilized in other types of prostheses beyond auditory prostheses.

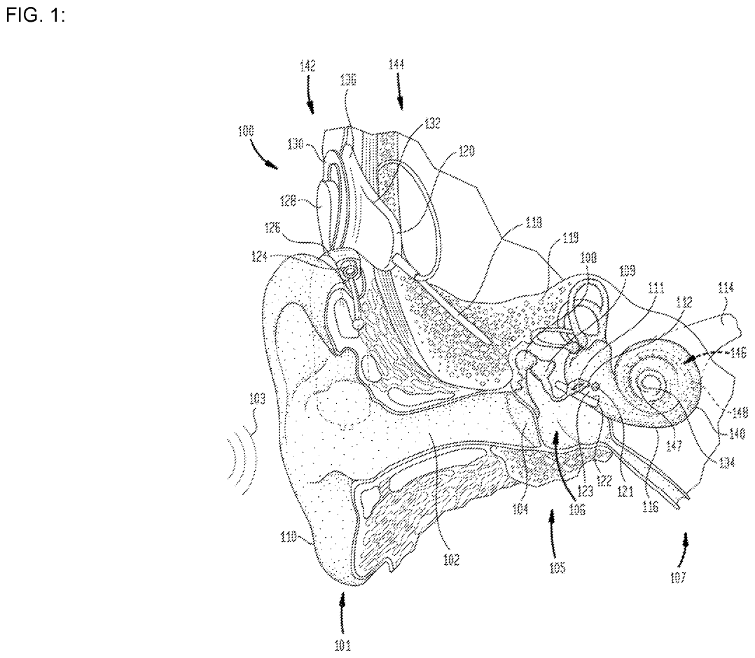

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1 with a subcutaneously implantable microphone assembly, as described more fully herein. In certain implementations, the example cochlear implant auditory prosthesis 100 of FIG. 1 can be in conjunction with a reservoir of liquid medicament as described herein.

As shown in FIG. 1, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

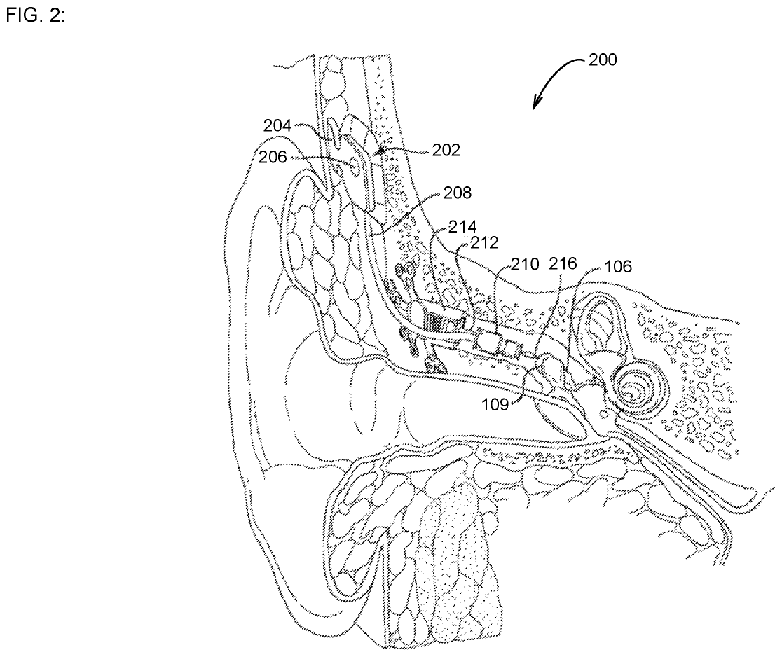

FIG. 2 schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 2 comprises a biocompatible implantable assembly 202 (e.g., comprising an implantable capsule) located subcutaneously (e.g., beneath the recipient's skin and on a recipient's skull). While FIG. 2 schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 2, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1 and 2 can comprise an implantable microphone assembly, such as the microphone assembly 206 shown in FIG. 2. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 2 is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

The example auditory prostheses 100 shown in FIG. 1 utilizes an external microphone 124 and the auditory prosthesis 200 shown in FIG. 2 utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1 and 2 are merely illustrative.

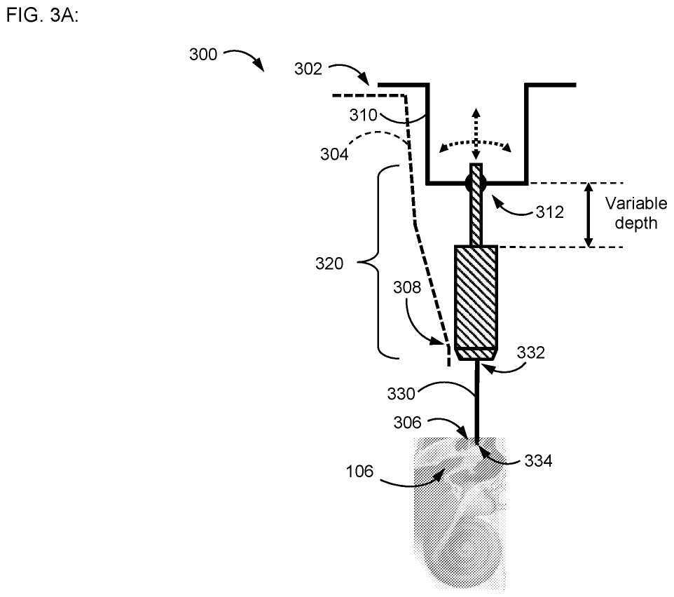

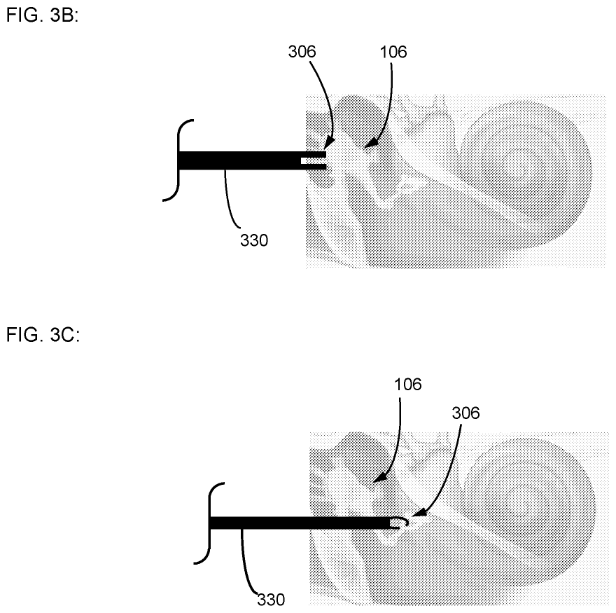

FIG. 3A schematically illustrates an example prior middle ear transducer assembly 300 that are based on a two-point fixation concept with one fixation point 302 at a surface of the recipient's skull 304 and a second fixation point 306 at a middle ear target (e.g., an ossicle 106; incus 109). The transducer assembly 300 bridges the physical gap between the two fixation points 302, 306 and comprises a fixation element 310 (e.g., bracket) that is affixed to the recipient's skull 304 and a transducer 320 in mechanical communication with the fixation element 310. The transducer 320 comprises a connection apparatus 330 (e.g., connection apparatus 216) having a first end 332 in mechanical communication with the transducer 320 and a second end 334 in mechanical communication with the middle ear target. The connection apparatus 330 is configured to conduct mechanical vibrations from the transducer 320 to the middle ear target.

The transducer assembly 300 further includes a linear motion mechanism (not shown) (e.g., z-adjustment microdrive and compression unit) that is configured to mechanically couple the transducer 320 to the fixation element 310 and to controllably adjust a linear position (e.g., depth) of the transducer 320 (denoted in FIG. 3A by a vertical dotted line) allowing for a z-adjustment microdrive movement of about 4 to 5 millimeters, in addition to a gross translation of the transducer 320 sliding through the compression unit (e.g., by a distance of about 4 to 5 millimeters). In addition, the transducer assembly 300 further comprises a rotatable coupler 312 configured to adjust an angle of the transducer 320 relative to the fixation element 310 (denoted in FIG. 3A by a curved dotted line).

The first end 332 of the connection apparatus 330 (e.g., connection apparatus 216) can comprise a solid first rod extending from a front portion of the transducer 320 and a hollow tube welded onto the first rod, forming a blind hole (e.g., 2 millimeters deep) configured to receive a first end of a solid second rod, the second rod comprising a second end that is the second end 334 of the connection apparatus 330. During an implantation process for the transducer assembly 300, the transducer 320 is positioned (e.g., at a depth selected to avoid the transducer 320 from contacting or being interfered by other bone portions 308 of the skull 304). A length measurement is made (e.g., using a template device) to determine a distance between the transducer 320 and the middle ear target (e.g., a distance between an inner surface of the blind hole of the transducer 320 into which the first end of the second rod is to be inserted), and the second rod is cut to an appropriate length using a cutting tool (e.g., on the operating room table), after which the first end of the second rod is positioned and affixed to the transducer 320 (e.g., the blind hole crimped onto the first end of the second rod), the transducer 320 is inserted and fixed to the fixation element 310, and the second end of the second rod is attached to the middle ear target. This process risks errors with the handling, cutting, and positioning of the second rod. Using connection apparatus 330 with pre-fixed lengths (e.g., a “one size fits all”) may not be recommended as adequate statistical data of the middle ear variability would be required and would result in design compromises. In addition, a pre-fixed connection apparatus 330 would be logistically complex to accommodate for the middle ear coupling variant options. FIGS. 3B and 3C schematically illustrate the second end 332 of the connection apparatus 330 having a structure configured to be affixed to the ossicles 106 (e.g., in a manner compatible with use in addressing SNHL and MHL, respectively) in accordance with certain implementations described herein.

As described above, in the two-point fixation concept as schematically illustrated in FIG. 3A, the connection apparatus 330 is typically affixed to the active portion (e.g., transducer) of the implant after the active portion has been implanted. If there is a problem during this portion of the implantation process, the active portion can be damaged (e.g., become unusable), resulting in undesirable effort to remove the active portion and replace it with an undamaged active portion. For high power actuators (e.g., higher powers than for a middle ear actuator) that are affixed to a portion of the skull and have a connection apparatus 330 affixed to a stimulation target (e.g., otic capsule), there is a risk of having parallel vibration paths to the cochlea, resulting in harmonic distortion and/or other sound quality issues and potentially resulting in vibration losses and/or transmission variability.

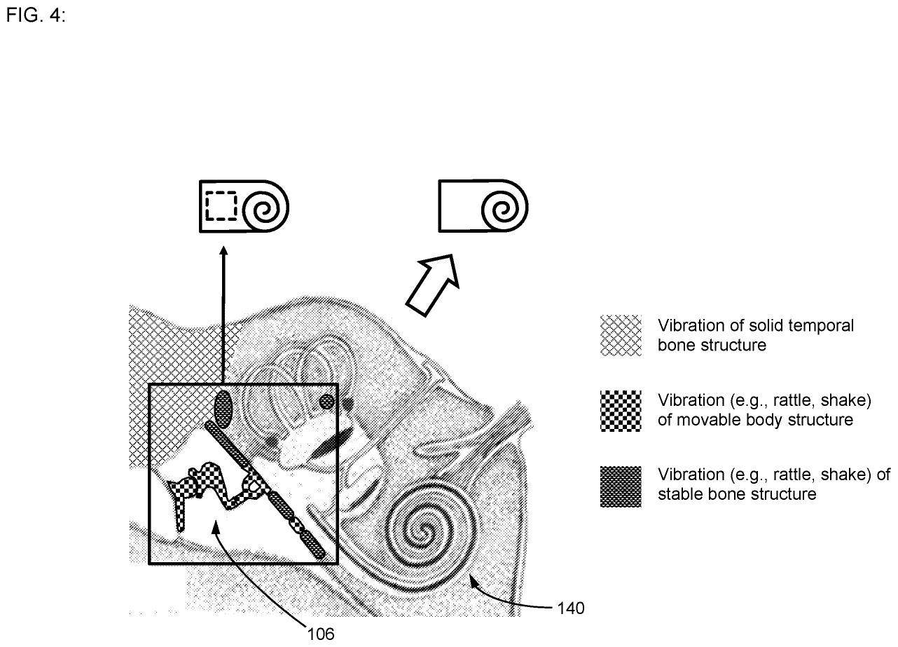

FIG. 4 schematically illustrates various types of vibration stimulation that can be provided to various structures in the cochleovestibular region in accordance with certain implementations described herein. A first type of stimulation provides vibrations to solid bone structure. For example, a bone-anchored transducer can provide vibrations indicative of sound to solid temporal bone tissue which transmits the vibrations to other portions of the auditory system (e.g., to the cochlea 140 while circumventing the middle ear portion of the auditory system). A second type of stimulation provides vibrations (e.g., rattle; shake) to movable body structures (e.g., ossicles 106, various fenestrae, perilymph) of the auditory system. A third type of stimulation provides vibrations (e.g., rattle; shake) to stable bone structure of the auditory system on or close to the cochlea 140 (e.g., otic capsule, promontory 123, lateral semicircular canal). Each of these types of vibration stimulation correspond to different ranges of mechanical impedance of the stimulation target (e.g., the first type of stimulation corresponds to high mechanical impedance, the second type of stimulation corresponds to low mechanical impedance, and the third type of stimulation corresponds to mechanical impedance between that of the first and second types). FIG. 4 also schematically illustrates two symbols which correspond to the whole middle and inner ear (e.g., cochleovestibular) regions and to the portion of the middle and inner ear regions that comprise structures compatible with the second and third types of stimulation in accordance with certain implementations described herein.

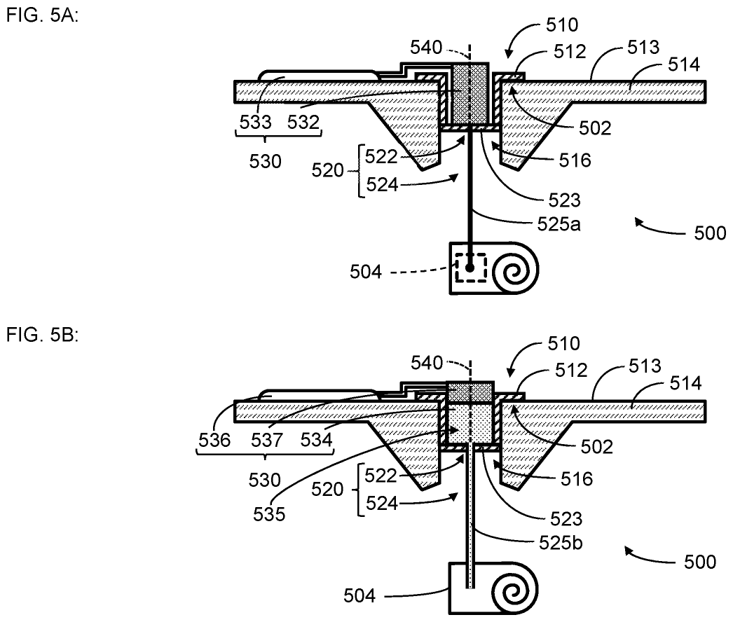

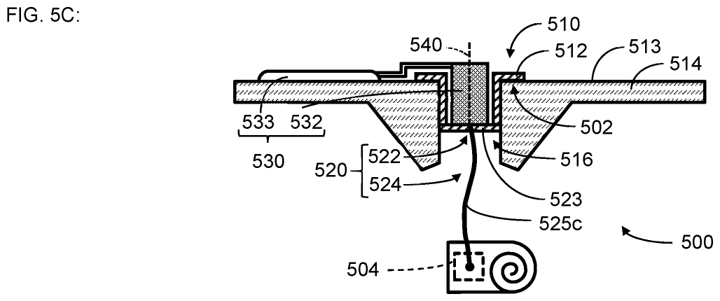

FIGS. 5A-5C schematically illustrate various example apparatus 500 in accordance with certain implementations described herein. The example apparatus 500 of FIGS. 5A-5C are configured to be affixed to a first location 502 on or within the recipient's body and to be in operative communication with a second location 504 of the recipient's body (e.g., a target portion of the recipient's body; in an inner ear region; in a middle ear region; within a cochleovestibular region; on a cochlea 140), the first location 502 spaced from the second location 504. The example apparatus 500 is also configured to receive an active component (e.g., transducer; actuator; microphone; reservoir; pump) configured to be in operative communication with the second location 504 via the apparatus 500.

The example apparatus 500 shown in FIGS. 5A-5C comprises a first portion 510 (e.g., fixation portion; fixation element; fixation bracket; fixture) configured to be implanted at the first location 502 and a second portion 520 (e.g., elongate portion; connection apparatus; vibration transmitter; elongate fluid conduit) configured to be implanted on or within the recipient's body and extending at least partially through the first portion 510. In certain implementations, the first portion 510 and the second portion 520 are components of a unitary structure (e.g., the second portion 520 is attached to the first portion 510 while the first portion 510 is being implanted at the first location 502), while in certain other implementations, the second portion 520 is separate from the first portion 510 while the first portion 510 is being implanted at the first location 502 and the second portion 520 is affixed to the first portion 510 after the first portion 510 has been implanted at the first location 502.

In certain implementations, the first portion 510 comprises a fixture 512 (e.g., fixation bracket) configured to be affixed at the first location 502 (e.g., to a surface 513 of a skull bone 514 of the recipient's body). For example, as schematically illustrated by FIGS. 5A-5C, the surface 513 can comprise an outer surface (e.g., top surface) of the skull bone 514 of the recipient and the fixture 512 is configured to extend at least partially within a region (e.g., channel 516) extending through the skull bone 514 of the recipient. For another example, the first location 502 can comprise another surface of the recipient's body (e.g., a machined surface of the skull bone 514, an example of which is an inner surface of the channel 516; a bottom surface of the skull bone 514).

In certain implementations, the second portion 520 comprises an orifice 522 and an elongate element 524 extending through the orifice 522 and configured to be in mechanical communication with the second location 504. The elongate element 524 of certain implementations comprises titanium or titanium alloy and can have a length in a range of 10 millimeters to 40 millimeters (e.g., in a range of 20 millimeters to 30 millimeters). As schematically illustrated by FIGS. 5A-5C, the second portion 520 of certain implementations is configured to be positioned at least partially within the channel 516 and comprises a plate 523 comprising the orifice 522 extending therethrough.

In certain implementations, the elongate element 524 is configured to be moved (e.g., slid; screwed) within the orifice 522 to adjust the position of the elongate element 524 relative to the orifice 522 and/or the plate 523 (e.g., to adjust a distance that the elongate element 524 extends out of the orifice 522 towards the second location 504). For example, the elongate element 524 can be moved (e.g., slid; screwed) within the orifice 522 along a distance within a range of 0.1 millimeter to 25 millimeters (e.g., 0.1 millimeter to 1 millimeter). In certain implementations, the elongate element 524 is also configured to be tilted within the orifice 522 and/or bent to adjust the orientation (e.g., angle) of the elongate element 524 relative to the orifice 522 and/or the plate 523 (e.g., to adjust an angle at which the elongate element 524 extends out of the orifice 522 towards the second location 504).

The second portion 520 of certain implementations is configured to be mechanically coupled to a transducer 532. In FIGS. 5A and 5C, the elongate element 524 comprises a rod 525a or wire 525c, respectively, having an outer diameter (e.g., in a range of 0.1 millimeter to 1 millimeter; in a range of 0.1 millimeter to 0.2 millimeter), a first end portion (e.g., a proximal end portion) configured to be in mechanical communication with the transducer 532, and a second end portion (e.g., a distal end portion) configured to be in mechanical communication with (e.g., contact) the second location 504 (e.g., structures compatible with the second and third types of stimulation of the auditory system). For example, a distal end of the elongate element 524 can be configured to be in mechanical communication with the ossicles 106 (see, e.g., FIGS. 3A-3C; comprising adhesive; comprising a clip configured to be slid onto the incus 109 while the elongate element 524 is adjusted into position during implantation). In certain implementations in which the elongate element 524 is configured to be in mechanical communication with the cochlea 140, a distal end of the elongate element 524 comprises an adapter plate or other type of bone anchor configured to be glued or otherwise affixed to the cochlea 140 (e.g., a round window, an oval window, an artificial window, a natural fenestrae, an artificial fenestrae, a promontory). In FIG. 5A, the rod 525a can be sufficiently rigid for vibrations generated by the transducer 532 (e.g., actuator; force-driven actuator; actuator 210 of a middle ear auditory prosthesis 200) to propagate to the second location 504. In FIG. 5C, the wire 525c can be flexible while allowing vibrations at the second location 504 (e.g., ossicles 106) to propagate to the transducer 532 (e.g., microphone; tubular microphone assembly). The microphone can be amplitude-driven by the wire 525c for maximum mobility and/or optimal sensitivity to movement of the ossicles 106.

In FIG. 5B, the elongate element 524 comprises an elongate fluid conduit 525b (e.g., tube) at least partially within the first portion 510 and configured to be in fluidic communication with the second location 504 (e.g., in the middle and/or inner ear regions). The fluid conduit 525b can be hollow and cylindrical with an inner diameter (e.g., in a range of 0.1 millimeter to 1 millimeter). The second portion 520 of certain implementations is configured to be in fluidic communication with a reservoir 534 configured to contain at least one liquid medicament 535 (see, e.g., FIG. 5B). For example, the fluid conduit 525b can comprise a first end portion (e.g., a proximal end portion) configured to be in fluidic communication with the reservoir 534 and a second end portion (e.g., a distal end portion) configured to be in fluidic communication with the second location 504, the second location 504 spaced from the first location 502 (e.g., the first location 510 is outside an inner ear region and a middle ear region of the recipient's body and the second location 520 is within the inner and/or middle ear region). In certain implementations, a reservoir 534 emptied during use can be re-filled and/or removed and exchanged with a filled reservoir 534 while the fluid conduit 525b remains in place (e.g., undisturbed).

In certain implementations, the first portion 510 has a longitudinal axis 540 along which the first portion 510 extends at least partially through tissue at the first location 502 (e.g., the skull bone 514). As schematically illustrated by FIGS. 5A-5C, in certain implementations, the longitudinal axis 540 is substantially aligned with the second location 504 and the elongate element 524 extends from the first portion 510 to the second location 504 along a direction substantially aligned with the longitudinal axis 540 (e.g., in a direction substantially perpendicular to the plate 523). In certain other implementations, the longitudinal axis 540 of the first portion 510 is not substantially aligned with the second location 504 (e.g., the longitudinal axis 540 is offset from a direction from the first portion 510 to the second location 504), and the elongate element 524 extends from the first portion 510 to the second location 504 along a direction that is at a non-zero angle relative to the longitudinal axis 540 (e.g., in a direction substantially non-perpendicular to the plate 523). In certain such implementations, in addition to sliding and/or screwing the elongate element 524 within the orifice 522, adjusting the second portion 520 comprises adjusting an angle of the elongate element 524 relative to the longitudinal axis 540 of the first portion 510 (e.g., bending the elongate element 524).

In certain implementations, as schematically illustrated by FIGS. 5A-5C, the apparatus 500 comprises a third portion 530 configured to be placed in operative communication with the first portion 510 and/or the second portion 520 after the second portion 520 is placed in operative communication with the second location 504. For example, as schematically illustrated by FIGS. 5A and 5C, the third portion 530 comprises a transducer 532 and a controller 533. In FIG. 5A, the transducer 532 comprises an actuator configured to generate, in response to control signals from the controller 533, mechanical vibrations (e.g., vibrational energy) that propagate from the actuator, along (e.g., through) the elongate element 524, to the second location 504. In FIG. 5C, the transducer 532 comprises a microphone configured to generate and transmit signals to the controller 533, the signals indicative of mechanical vibrations (e.g., vibrational energy) received by the microphone after propagating along (e.g., through) the elongate element 524 from the second location 504. In certain other implementations, as schematically illustrated by FIG. 5B, the third portion 530 comprises a reservoir 534, a controller 536, and a flow control element 537 (e.g., valve; pump) configured to respond to control signals from the controller 536 to controllably flow the fluid (e.g., the at least one medicament 535) from the reservoir 534, along (e.g., through) the elongate element 524, to the second location 504 (e.g., near the cochlea 140; at or in the cochlea 140). While FIGS. 5A-5C show the third portion 530 (e.g., transducer 532; reservoir 534 and/or flow control element 537) extending above the first portion 510 (e.g., fixture 512), in certain other implementations, the third portion 530 is fully recessed within the first portion 510.

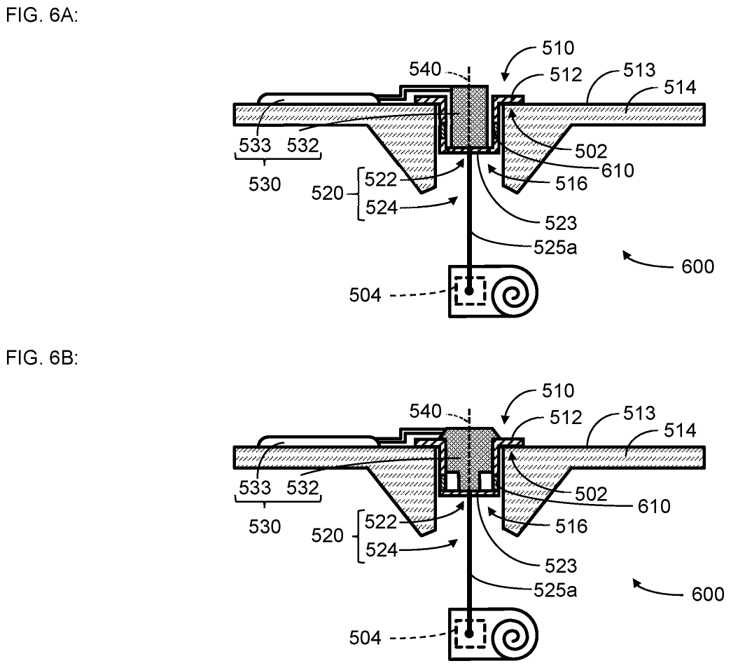

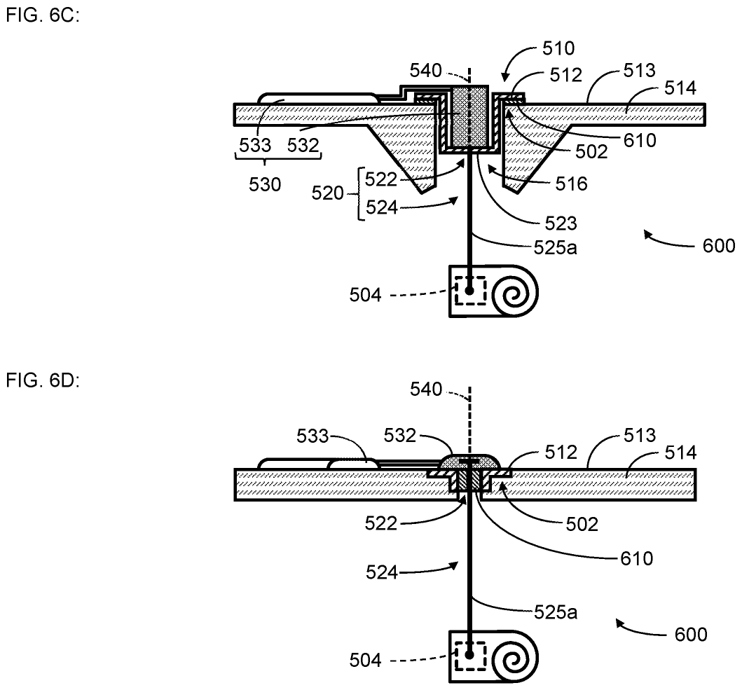

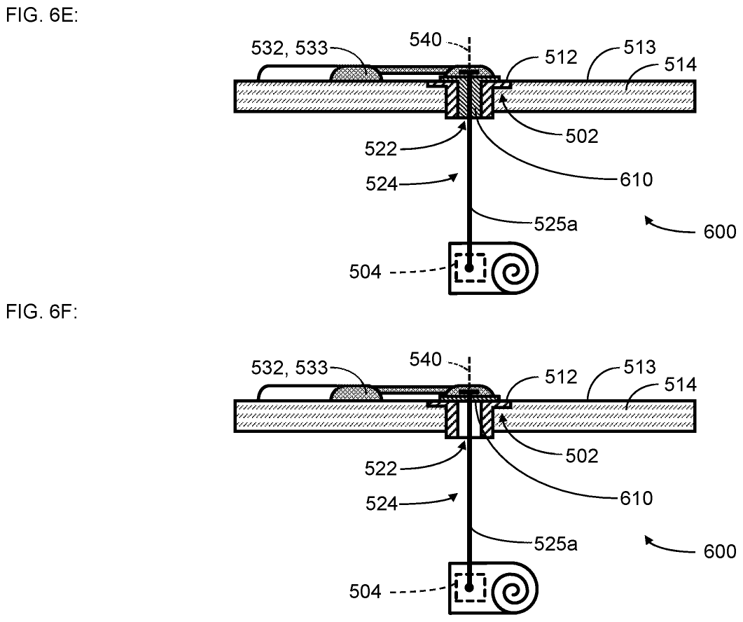

FIGS. 6A-6F schematically illustrate various example apparatus 600 in accordance with certain implementations described herein. The example apparatus 600 of FIGS. 6A-6F comprise a mechanically compliant portion 610 configured to inhibit (e.g., prevent) escape of the vibrational energy from the second portion 520 to portions of the recipient's body spaced from the second location 504 (e.g., target portion of the recipient's body). For example, the mechanically compliant portion 610 can comprise at least one flexible material (e.g., rubber; plastic) and/or at least one resilient member (e.g., spring) configured to provide vibration isolation. In certain implementations in which the transducer 532 generates mechanical vibrations (e.g., vibrational energy) to propagate along (e.g., through) the elongate element 524 to the second location 504, the mechanically compliant portion 610 is configured to substantially inhibit (e.g., prevent) the vibrations from propagating along other pathways to reach the second location 504 (e.g., the vibrations generated by the transducer 532 can reach the second location 504 only by propagating along the second portion 520).

In certain other implementations in which the transducer 532 (e.g., microphone) receives mechanical vibrations (e.g., vibrational energy) that propagate along (e.g., through) the elongate element 524 from the second location 504, the mechanically compliant portion 610 is configured to substantially inhibit (e.g., prevent) other vibrations from other sources (e.g., body noise) from reaching the transducer 532. For example, the mechanically compliant portion 610 can be used with a fully implantable system comprising an implantable microphone and can be configured to inhibit (e.g., prevent) alternative vibration paths to the cochlea 140 and/or a feedback path to the implanted microphone. Certain implementations comprise both an actuator and a microphone seated in the same apparatus 600 with both the actuator and the microphone sufficiently vibrationally isolated from one another.

As schematically illustrated by FIGS. 6A and 6B, the mechanically compliant portion 610 of certain implementations is positioned between the first portion 510 (e.g., fixation portion) and the second portion 520 (e.g., elongate portion). In certain other implementations, the mechanically compliant portion 610 is integrated in the orifice 522 between the elongate element 524 and a plate 523, and the transducer 532 can be affixed to the elongate element 524. As schematically illustrated by FIG. 6C, the mechanically compliant portion 610 can be positioned between the first portion 510 and the tissue at the first location 502 (e.g., skull bone 514).

In FIG. 6A, the transducer 532 is within but does not contact the first portion 510 while in mechanical communication with the second portion 520. In FIG. 6B, the transducer 532 is within and in mechanical communication with the first portion 510 (e.g., screwed into the first portion 510) while in mechanical communication with the second portion 520. In certain such implementations, the transducer 532 is pre-loaded to apply a force onto the second portion 520. While FIGS. 6A-6C schematically illustrate example configurations in which the first portion 510 and the second portion 520 are spaced from an inner surface of the channel 516, in other example configurations, one or both of the first portion 510 and the second portion 520 can contact a portion of the bone tissue extending between the first location 502 and the second location 504. For example, as schematically illustrated by FIGS. 6D-6F, the apparatus 600 can comprise an osseointegrated fixture 512 contacting bone tissue below the surface 513 and the elongate element 524 can extend through an orifice 522 of the fixture 512 and through a channel 516 through the skull bone 514. In FIG. 6D, the transducer 532 is above and in mechanical communication with the fixture 512. In FIGS. 6E and 6F, the transducer 532 is displaced laterally from the fixture 512 and the mechanically compliant portion 610 is configured to vibrationally isolate the elongate element 524 from the fixture 512. In FIG. 6E, the mechanically compliant portion 610 extends through the fixture 512, while in FIG. 6F, the mechanically compliant portion 610 is on top of the fixture 512. In certain implementations, the fixture 512 can be recessed into the surface 513 of the skull bone 514 (e.g., a top surface of the fixture 512 can be below or flush with the surface 513 of the skull bone 514; see, e.g., FIGS. 6D-6F), while in certain other implementations, the top surface of the fixture 512 can extend above the surface 513.

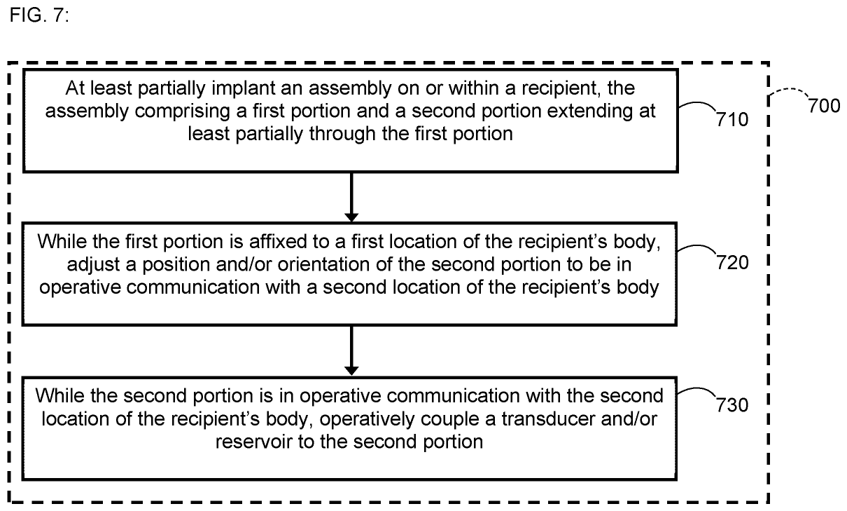

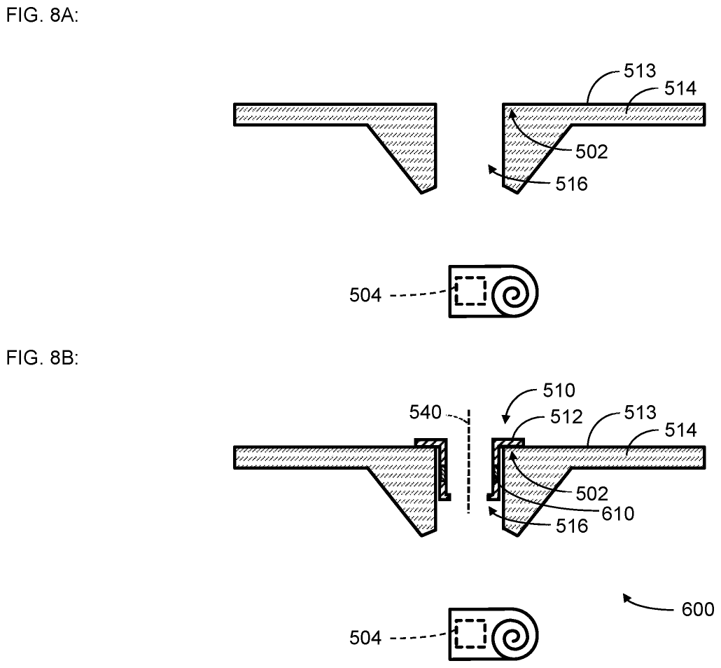

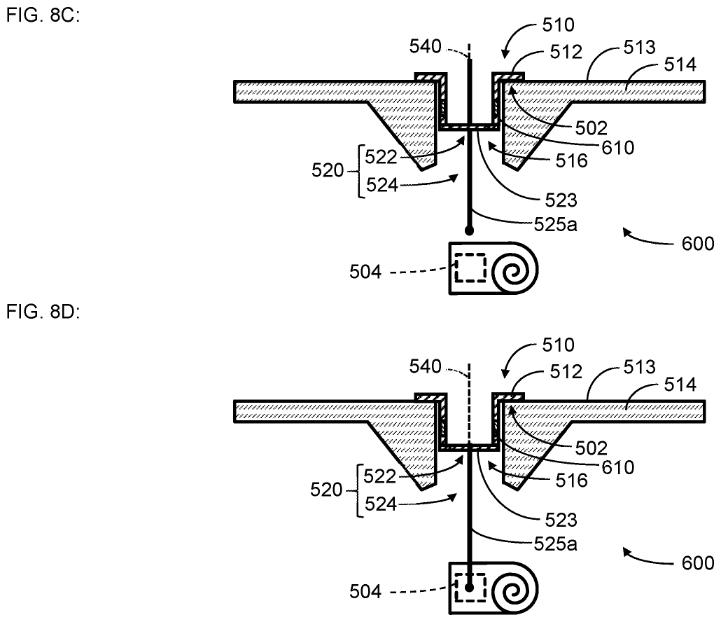

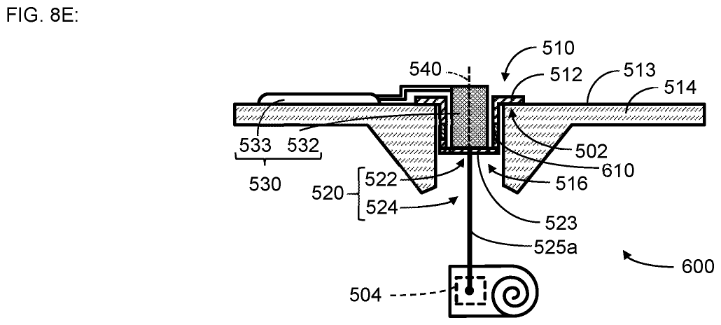

FIG. 7 is a flow diagram of an example method 700 in accordance with certain implementations described herein and FIGS. 8A-8E schematically illustrate various stages of an assembly (e.g., apparatus 500; apparatus 600) in accordance with certain implementations described herein during the example method 700. While the example method 700 is described herein by referring to the example apparatus 500, 600 of FIGS. 5A-5C and 6A-6F, other apparatuses are also compatible with the example method 700 in accordance with certain embodiments described herein. For example, the method 700 described herein can be applied to any of a variety of implantable medical devices.

In certain implementations, the method 700 comprises drilling a channel 516 through a portion of the bone tissue (e.g., skull bone 514), the channel 516 extending from a top surface (e.g., surface 513) of the bone tissue. FIG. 8A schematically illustrates the surface 513, skull bone 514, and channel 516, with the channel 516 extending along a direction from the first location 502 to the second location 504.

In an operational block 710, the method 700 comprises at least partially implanting an assembly on or within a recipient's body. As schematically illustrated by FIG. 8B, the assembly comprises a first portion 510 comprising a fixture 512 with a mechanically compliant portion 610, as described herein with regard to FIG. 6A, although other first portions 510 are also compatible with certain implementations described herein (e.g., a first portion 510 that does not comprise a mechanically compliant portion 610 and/or that is not mechanically coupled directly or indirectly to a mechanically compliant portion 610; see, e.g., FIGS. 5A-5C). In certain implementations, said at least partially implanting comprises affixing the first portion 510 to the first location 502. For example, one or more bone screws and/or an adhesive applied to one or both of the first portion 510 and the bone tissue (e.g., skull bone 514) can be used to affix the first portion 510 at the first location 502. In other examples in which the first portion 510 comprises a fixture 512, the fixture 512 can be screwed into the bone tissue at the first location 502 (see, e.g., FIGS. 6D-6F).

In certain implementations, at least partially implanting the assembly further comprises affixing the second portion 520 to the first portion 510. For example, as schematically illustrated in FIG. 8C, the second portion 520 can comprise an orifice 522 (e.g., extending through a plate 523) and an elongate element 524 configured to extend through the orifice 522. In certain implementations (see, e.g., FIGS. 8B and 8C), the second portion 520 is initially separate from the first portion 510 and is configured to be affixed to the first portion 510 (e.g., by crimping or otherwise mechanically deforming one or both of the first portion 510 and the second portion 520; using mating portions of the first portion 510 and the second portion 520 and/or other fixation elements, such as screws and/or adhesive applied to one or both of the first portion 510 and the second portion 520) after the first portion 510 has been affixed to the first location 502 or while the first portion 510 is in the process of being affixed to the first location 502. In certain other implementations, the second portion 520 is integral with the first portion 510 (see, e.g., FIG. 6C-6F).

In certain implementations, the elongate element 524 is initially separate from the orifice 522 and is configured to be inserted within the orifice 522 after the plate 533 or other component comprising the orifice 522 has been affixed to the first portion 510 or while the plate 533 is in the process of being affixed to the first portion 510. In certain other implementations, the elongate element 524 is inserted within the orifice 522 prior to the process of affixing the plate 533 or other component comprising the orifice 522 to the first portion 510.

In an operational block 720, the method 700 further comprises, while the first portion 510 is affixed to the first location 502, adjusting the second portion 520 (e.g., adjusting a position and/or orientation of the second portion 520) to be in operative communication with the second location 504 of the recipient's body. For example, adjusting the second portion 520 can be performed after the first portion 510 has been affixed to the first location 502, while the first portion 510 is in the process of being affixed to the first location 502, after the second portion 520 has been affixed to the first portion 510, or while the second portion 520 is in the process of being affixed to the first portion 510. In certain implementations in which the second portion 520 is initially separate from the first portion 510, adjusting the second portion 520 can be performed prior to affixing the second portion 520 to the first portion 510.

In certain implementations, as schematically illustrated by FIG. 8D, adjusting the second portion 520 comprises moving (e.g., sliding; screwing) and/or angling the elongate element 524 within the orifice 522 to place the distal end of the elongate element 524 in mechanical communication with the second location 504 while the elongate element 524 remains within the orifice 522. For example, the elongate element 524 can be slidably adjusted (e.g., along an axial direction of the elongate element 524; along the longitudinal axis 540) relative to the orifice 522. The linear position or depth of the distal end of the elongate element 524 can be adjusted along a distance within a range of 0.1 millimeter to 25 millimeters (e.g., 0.1 millimeter to 1 millimeter) such that the elongate element 524 spans a desired distance between the first portion 510 and the second location 504 (e.g., a desired coupling distance). In certain such examples, the elongate element 524 can also be angled (e.g., rotated; bent) relative to the longitudinal axis 540 of the first portion 510 such that the distal end of the elongate element 524 reaches the second location 504.

In certain implementations, adjusting the second portion 520 further comprises affixing the elongate element 524 to the component comprising the orifice 522 such that the second portion 520 is no longer adjustable relative to the orifice 522. For example, one or both of the elongate element 524 and the component comprising the orifice 522 can be crimped or otherwise mechanically deformed and/or affixed to one another by adhesive.

In certain implementations, as schematically illustrated by FIG. 8D, adjusting the second portion 520 can comprise cutting and removing a portion of the elongate element 524 from within a region on an opposite side of the first portion 510 from the second location 504. For example, cutting and removing the portion of the elongate element 524 can be performed after moving (e.g., sliding; screwing) and/or angling the elongate element 524 within the orifice 522, while the elongate element 524 is in the process of being moved and/or angled within the orifice 522, after affixing the elongate element 524 to the component comprising the orifice 522, or while the process of affixing the elongate element 524 to the component comprising the orifice 522 is being performed (e.g., using a tool configured to both crimp and cut). As schematically illustrated by FIG. 8D, the first portion 510 and the second portion 520 can be implanted and in operative communication with the second location 504 prior to the transducer 532 and/or reservoir 534 being implanted. By cutting and removing a portion of the elongate element 524 on an opposite side of the orifice 522 from the second location 504, certain implementations described herein can reduce the risk of losing the cut portion and/or of measuring and/or cutting errors.

In an operational block 730, the method 700 further comprises, while the second portion 520 is in operative communication with the second location 504 of the recipient's body, operatively coupling (e.g., directly or indirectly) the second portion 520 with a third portion 530 of the assembly (e.g., a transducer 532 and/or a reservoir 534 configured to contain fluid). For example, operatively coupling the second portion 520 with the third portion 530 can be performed after the second portion 520 has been placed in operative communication with the second location 504 or while the second portion 520 is in the process of being placed in operative communication with the second location 504. In certain implementations, operatively coupling the second portion 520 with the third portion 530 comprises affixing the third portion 530 to the first portion 510 and/or the second portion 520 (e.g., by crimping or otherwise mechanically deforming one or more of the first portion 510, second portion 520, and third portion 530; using mating portions of the third portion 530 with mating portions of the first portion 510 and/or the second portion 520 and/or other fixation elements, such as screws and/or adhesive applied to one or more of the first portion 510, second portion 520, and third portion 530) such that the third portion 530 is in mechanical communication with the second portion 520. For example, a transducer 532 of the third portion 530 can be affixed to the proximal end of the elongate element 524 (e.g., rod 525a; wire 525c) or to a component comprising the orifice 522 that is affixed to the elongate element 524. For another example, a reservoir 534 of the third portion 530 can be affixed to the proximal end of the elongate element 524 (e.g., fluid conduit 525b) such that the reservoir 534 is in fluidic communication with the second portion 520 (e.g., and with the second location 504).

Certain implementations described herein provide simplified implantation procedures and/or tools, thereby reducing the probability of errors or mishaps during the assembly implantation process resulting from the handling, cutting, and positioning of the elongate element 524 between the transducer and sensitive structures of the recipient's body. For example, the manipulation of the elongate element 524 is reduced, thereby reducing the risk of losing or damaging the elongate element 524, as well as the risk of measuring or cutting errors.

Certain implementations described herein enable various extension coupling variant options without logical complexity. For example, an elongate element 524 can be pre-slid into the assembly, but the elongate element 524 can be easily replaced in the operating room by another elongate element if desired. Certain implementations described herein enable similar surgical procedures and configurations to be used for assemblies configured to address SNHL and assemblies configured to address MHL, thereby resulting in more consistent surgeries for a wider range of surgeons and more consistent outcomes.

Certain implementations described herein reduce the risk of mechanical interference (e.g., contact between the assembly and surrounding bone) by having only the elongate element 524 linearly translated into position, prior to the transducer 532 being implanted. In addition, assemblies of certain implementations described herein do not include a linear motion mechanism (e.g., z-direction microdrive and compression unit) for moving the transducer 532, thereby reducing complexity and expense (e.g., only including an axial rotational degree of freedom).

Certain implementations described herein provide simplification of the surgical process of implantation, reduce logistic complexity, and are applicable to all extension coupling variant options. By having the elongate element 524 extend downwards (e.g., into the cochleovestibular region) instead of the transducer 532, certain implementations reduce the risk of mechanical interference (e.g., inadvertent contact) of the actuator with the inner surfaces of the channel 516.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a first portion configured to be implanted at a first location on or within a recipient's body; and
a second portion configured to be implanted on or within the recipient's body and configured to be mechanically coupled to a transducer, the second portion comprising an orifice and an elongate element extending through the orifice, the elongate element configured to be in mechanical communication with a second location on or within the recipient's body, the second location spaced from the first portion, the first portion and the second portion configured to be implanted with the elongate element in mechanical communication with the second location prior to the second portion being mechanically coupled to the transducer.

2. The apparatus of claim 1, wherein the first portion is configured to extend at least partially within a channel or cavity in a skull bone of the recipient, and the second portion is configured to be positioned at least partially within the channel or cavity.

3. The apparatus of claim 1, wherein the second portion comprises a plate with the orifice extending therethrough and the elongate element comprises a rod or wire configured to contact the second location.

4. The apparatus of claim 1, wherein the elongate element is configured to be affixed within the orifice after being slidably adjusted within the orifice such that an end of the elongate element contacts the second location.

5. The apparatus of claim 1, wherein the first portion is configured to be affixed to a skull bone surface, and the second location is in an inner or middle ear region of the recipient's body.

6. The apparatus of claim 5, wherein the second location is on a cochlea and/or within a cochleovestibular region of the recipient's body.

7. The apparatus of claim 1, wherein the second portion is configured to be separate from the first portion while the first portion is being implanted at the first location and the second portion is configured to be affixed to the first portion after the first portion has been implanted at the first location.

8. The apparatus of claim 1, wherein the second portion is attached to the first portion while the first portion is being implanted at the first location.

9. The apparatus of claim 1, further comprising vibration isolation material between the first portion and the second portion and/or between the first portion and the first location.

10. The apparatus of claim 1, wherein the transducer is configured to generate mechanical vibrations that propagate through the elongate element to the second location.

11. The apparatus of claim 1, wherein the first portion has a longitudinal axis along which the first portion is configured to extend at least partially through bone tissue.

12. The apparatus of claim 11, wherein the elongate element extends substantially parallel to the longitudinal axis.

13. The apparatus of claim 12, wherein the elongate element extends along a direction at a non-zero angle relative to the longitudinal axis.

14. An apparatus comprising:
a fixation portion configured to be implanted on or within a recipient's body;
an elongate portion configured to be in mechanical communication with a transducer and a target portion of the recipient's body, the elongate portion extending at least partially through the fixation portion and configured to transmit vibrational energy from the transducer to the target portion and/or from the target portion to the transducer; and
at least one mechanically compliant portion between the fixation portion and the elongate portion, the at least one mechanically compliant portion configured to substantially inhibit escape of the vibrational energy from the elongate portion to portions of the recipient's body spaced from the target portion and/or to substantially inhibit vibrational energy not from the target portion from reaching the transducer.

15. The apparatus of claim 14, wherein the at least one mechanically compliant portion comprises at least one flexible material and/or at least one resilient member.

16. A method comprising:
at least partially implanting an assembly on or within a recipient's body, the assembly comprising a fixation portion and an elongate portion extending at least partially through the fixation portion, said at least partially implanting comprising affixing the fixation portion to a first location of the recipient's body;
while the fixation portion is affixed to the first location, adjusting a position and/or orientation of the elongate portion to be in operative communication with a second location of the recipient's body; and
while the elongate portion is in operative communication with the second location of the recipient's body, operatively coupling the elongate portion with a transducer and/or a reservoir configured to contain at least one medicament.

17. The method of claim 16, further comprising, during and/or after adjusting the position and/or orientation of the elongate portion, mechanically coupling the elongate portion to the fixation portion.

18. The method of claim 16, wherein operatively coupling the elongate portion with a transducer and/or a reservoir comprises affixing the transducer to the fixation portion and/or the elongate portion such that the transducer is in mechanical communication with the elongate portion.

19. The method of claim 16, wherein operatively coupling the elongate portion with a transducer and/or a reservoir comprises affixing the reservoir to the fixation portion and/or the elongate portion such that the reservoir is in fluidic communication with the elongate portion.

20. The method of claim 16, wherein affixing the elongate portion to the fixation portion comprises mechanically deforming one or both of the elongate portion and the fixation portion and/or applying an adhesive to one or both of the elongate portion and the fixation portion.

21. The method of claim 16, wherein at least partially implanting the assembly comprises affixing the fixation portion to the first location, the first location is at a skull bone surface, and the second location of the recipient's body is at an inner ear region; in a middle ear region, or within a cochleovestibular region of the recipient's body.

22. An apparatus comprising:
a fixation bracket configured to be affixed at a first location on or within a recipient's body; and
an elongate fluid conduit configured to be at least partially within the fixation bracket, the elongate fluid conduit comprising:
a first end portion configured to be in fluidic communication with a reservoir configured to contain fluid; and
a second end portion configured to be in fluidic communication with a second location on or within the recipient's body and spaced from the first location, the fixation bracket and the elongate fluid conduit configured to be implanted on or within the recipient's body prior to the reservoir being in fluidic communication with the first end portion.

23. The apparatus of claim 22, wherein the apparatus further comprises the reservoir and the fluid, and the fluid comprises at least one medicament.

24. The apparatus of claim 22, wherein the fixation bracket is configured to at least partially contain the reservoir.

25. The apparatus of claim 22, wherein the first location is outside an inner ear region and a middle ear region of the recipient's body and the second location is within the inner and/or middle ear region.
